(19) United States
(12) Patent Application Publication
(10) Pub. No.: US 2017/0209183 A1
(43) Pub. Date: Jul. 27, 2017

(54) SURGICAL IMPLANT AND METHOD OF USE

(71) Applicant: Warsaw Orthopedic, Inc., Warsaw, IN (US)

(72) Inventors: Jeffrey N. Wickham, Hernando, MS (US); Mark C. Dace, Collierville, TN (US); Alexander Vaccaro, Gladwyne, PA (US); Brian A. O'Shaughessy, Nashville, TN (US); Andrew Hecht, New York, NY (US)

(21) Appl. No.: 15/004,491

(22) Filed: Jan. 22, 2016

(51) Int. Cl. A61B 17/70

(52) U.S. Cl. CPC A61B 17/7035

(57) ABSTRACT

A spinal implant includes a first portion including a head. A second portion includes a threaded shaft having a length and a diameter. The threaded shaft includes a length to diameter ratio of less than 2.0. Systems and methods of use are disclosed.

### TECHNICAL FIELD

The present disclosure generally relates to medical devices for the treatment of spinal disorders, and more particularly to a surgical implant system including a bone fastener and a method for treating a spine.

### BACKGROUND

Spinal pathologies and disorders such as scoliosis and other curvature abnormalities; kyphosis; degenerative disc disease; disc herniation; osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, plates, connectors and vertebral rods are often used to provide stability to a treated region. These implants can redirect stresses away from a damaged or defective region while healing takes place to restore proper alignment and generally support the vertebral members. For example, the plates, connectors and/or rods may be attached via the fasteners to the exterior of one or more vertebral members. This disclosure describes an improvement over these prior technologies.

### SUMMARY

In one embodiment, a spinal implant for use with a surgical treatment is provided. The spinal implant includes a first portion including a head. A second portion includes a threaded shaft having a length and a diameter. The threaded shaft includes a length to diameter ratio of less than 2.0. In some embodiments; systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a perspective view of he components shown in FIG. 1:

FIG. 3 is a side view of the components shown in FIG. 1;

FIG. 4 is a side view of the components shown in FIG. 1;

FIG. 5 is a side view of the components shown in FIG. 1;

FIG. 6 is a side view of the components shown in FIG. 1;

FIG. 7 is an end view of the components shown in FIG. 1;

FIG. 8 is an end view of the components shown in FIG. 1;

FIG. 9 is a side, cross section view of the components shown in FIG. 1;

FIG. 10 shows a table of dimensions of components of one embodiment of a system in accordance with the principles of the present disclosure;

FIG. 11 is an axial view of vertebrae in connection with components of one embodiment of a system in accordance with the principles of the present disclosure;

FIG. 12 is an enlarged view of detail A shown in FIG. 11;

FIG. 13 is a side view of vertebrae in connection with components of one embodiment of a system in accordance with the principles of the present disclosure; and

FIG. 14 is an enlarged view of detail B shown in FIG. 13.

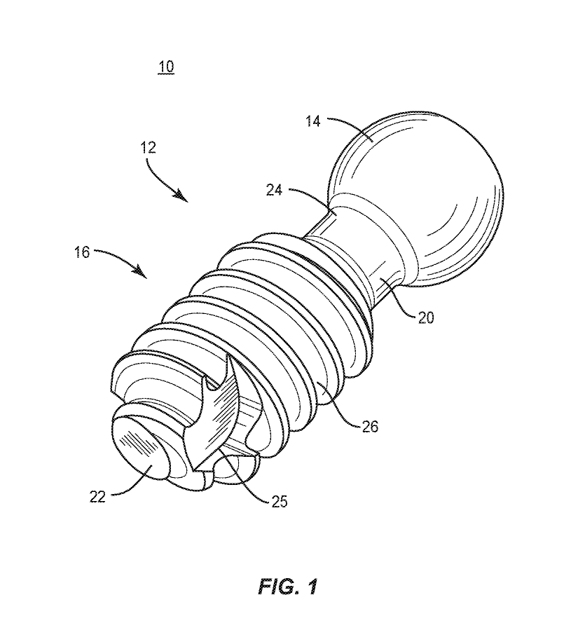

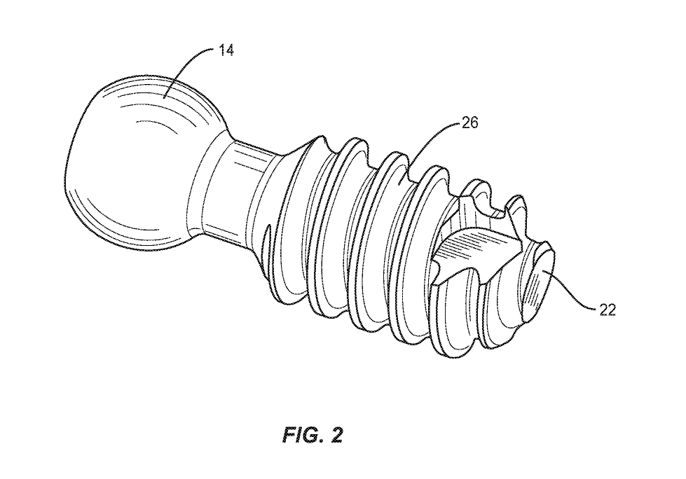

### DETAILED DESCRIPTION

The exemplary embodiments of a surgical system and related methods of use disclosed are discussed in terms of medical devices for the treatment of musculoskeletal disorders and more particularly, in terms of a spinal implant system including a bone screw and a method for treating a spine.

In some embodiments, the present surgical system includes a spinal implant comprising a bone screw having a selected length and a selected diameter. In some embodiments, the spinal implant is configured for a minimally invasive lateral mass screw placement. In some embodiments, the spinal implant includes a bone screw configured to reduce a risk of vertebral artery or nerve root injury. In some embodiments; the spinal implant includes a bone screw having a selected length and a selected diameter to facilitate lateral mass placement. In some embodiments, the spinal implant includes a bone screw having a length to diameter aspect ratio in a range of 2.0 or less, a diameter in a range of 4.5 millimeters (mm) to 8.0 mm and a length in a range of 5.0 mm to 10.0 mm. In some embodiments, the spinal implant includes a bone screw having a diameter in a range of 5.5 mm to 7.0 mm and a length in a range of 6.0 mm to 8.0 mm.

In some embodiments, the present surgical system includes a spinal implant comprising a bone screw that is employed with a surgical method that aligns the bone screw with a direction of a force application to maximize the pullout force from a trajectory standpoint to resist and/or prevent vertebral artery or nerve root injury. In some embodiments, the surgical method includes a step of aligning a bone screw along a straight-in screw trajectory and/or surgical pathway. In some embodiments, the bone screw includes an axis aligned with a direction of pullout force to provide an increased resistance to pullout.

In some embodiments, the present surgical system includes a spinal implant comprising a bone screw that is employed with a surgical method, such as, for example, a Roy-Camille technique to resist and/or prevent vertebral artery or nerve root injury. In some embodiments, the present surgical system includes a spinal implant comprising a bone screw that is employed with a surgical method to facilitate a safe, simple, secure fixation and less invasive procedure for lateral mass screw placement. In some embodiments, the present surgical system includes a spinal implant comprising a bone screw that is employed with a surgical method to prevent a breach of an anterior cortex of a lateral mass.

In some embodiments, the present surgical system includes a bone screw configured with a selected diameter and a selected bone screw length to provide an increased pullout force, In some embodiments, the selected diameter is based on a size of a lateral mass. In some embodiments, the bone screw can have a diameter in a range of 5.0 mm to 7.0 mm to maintain bone for good structural integrity.

In some embodiments, the bone screw has a tapered minor diameter. In some embodiments, the bone screw has a length of 8.0 mm. In some embodiments, the bone screw has a diameter of 5.0 mm. In some embodiments, the bone screw has a tapered tip. In some embodiments, the bone screw has a thread cutting element.

In some embodiments, the present surgical system includes a spinal implant comprising a bone screw having a selected diameter that is employed with a surgical method to facilitate cervical procedures. In some embodiments, the present surgical system includes a spinal implant comprising a bone screw having a length to diameter ratio in a range of 2.0 or less. In some embodiments, the bone screw has a diameter in a range of 4.5 mm to 8.0 mm and a length in a range of 5.0 mm to 10.0 mm. In some embodiments, the bone screw has a length in a range of 5.5 mm to 7.0 mm and/or a diameter in a range of 6.0 mm to 8.0 mm.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic, sacral and pelvic regions of a spinal column. The surgical system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

The following discussion includes a description of a surgical system including one or more spinal implants, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS, 1-9, there are illustrated components of a surgical system 10 including a spinal implant, such as, for example, a bone screw 12.

The components of surgical system 10 can be fabricated from biologically acceptable materials suitable for medical applications, including metals, synthetic polymers, ceramics and bone material and/or their composites. For example, the components of surgical system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

Bone screw 12 includes a portion, such as, for example, a head 14 and a portion, such as, for example a shaft 16. Head 14 includes a substantially spherical portion. Head 14 includes a tool engaging portion 18 configured to engage a surgical tool or instrument (not shown), as described herein. In some embodiments, portion 18 includes a hexagonal cross-section to facilitate engagement with a surgical tool or instrument. In some embodiments, head 14 may have alternative cross-sections, such as, for example, rectangular, polygonal, hexalobe, oval, or irregular. In some embodiments, portion 18 may have a cruciform, Phillips, square, polygonal or star cross sectional configuration configured for disposal of a correspondingly shaped portion of a surgical tool or instrument.

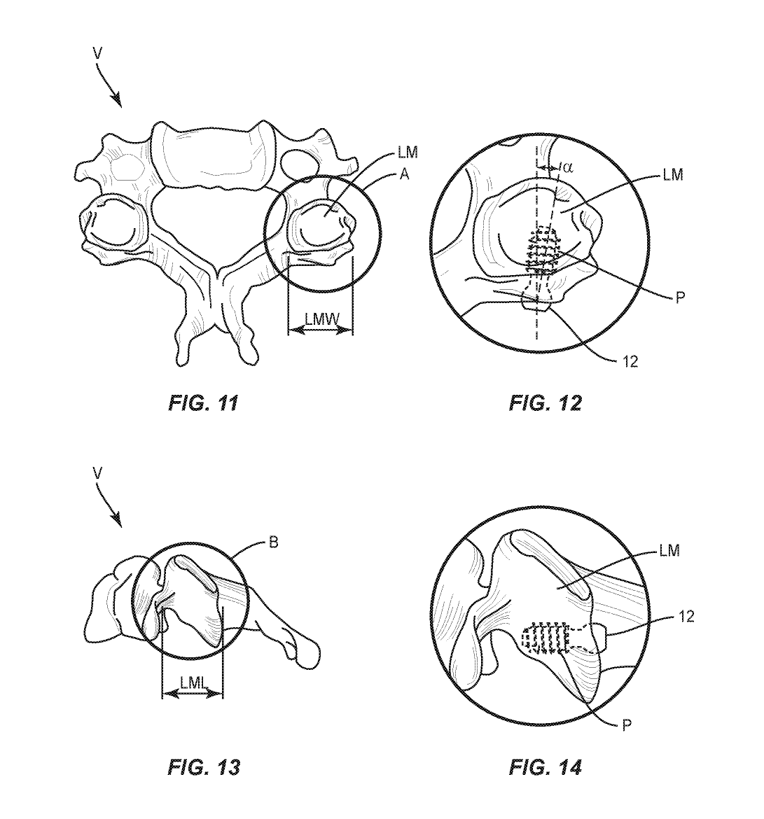

Shaft 16 extends along an axis X1. Shaft 16 includes an end 20 and an end 22. End 20 forms a section, such as, for example, a neck 24 with head 14. Neck 24 is disposed adjacent head 14. End 22 is configured to penetrate tissue, such as, for example, lateral mass tissue LM, as shown in FIGS. 11-14, as described herein. In some embodiments, shaft 16 is configured for disposal along a surgical pathway P, as shown in FIGS. 12 and 14, oriented at an angle α. In some embodiments, angle α is in a range of −5 through 15 degrees, and in some embodiments 0 through 10 degrees, relative to a direct posterior surgical approach, as described herein. In some embodiments, end 22 includes a tapered tip. In some embodiments, end 22 includes a blunt tip configured to facilitate resistance and/or prevent damage to surrounding tissue and/or nerves. In some embodiments, the blunt tip configuration can be employed to optimize bone purchase. In some embodiments, end 22 includes a thread cutting element, such as, for example, a groove 25 configured to facilitate engagement with tissue. In some embodiments, groove 25 is configured to penetrate tissue to facilitate engagement of a thread 30 with tissue, as described herein.

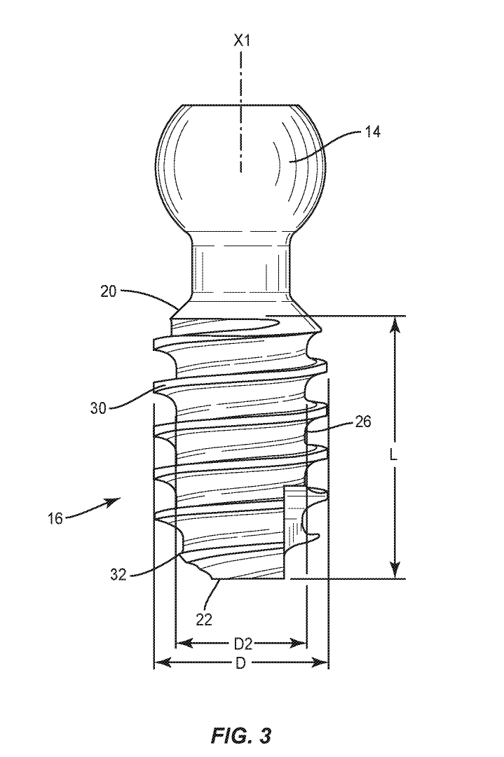

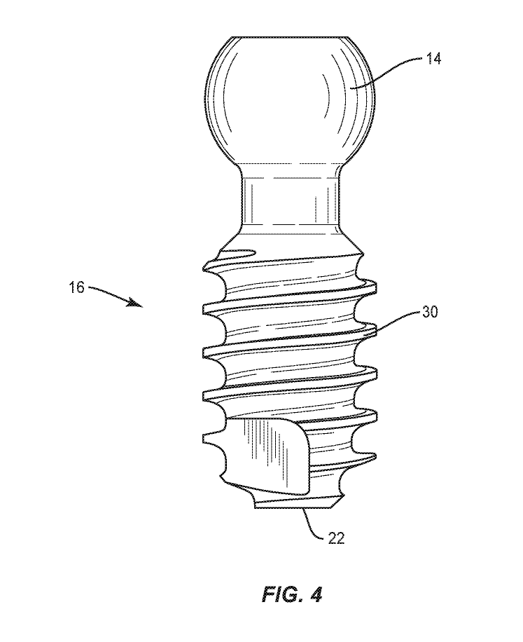

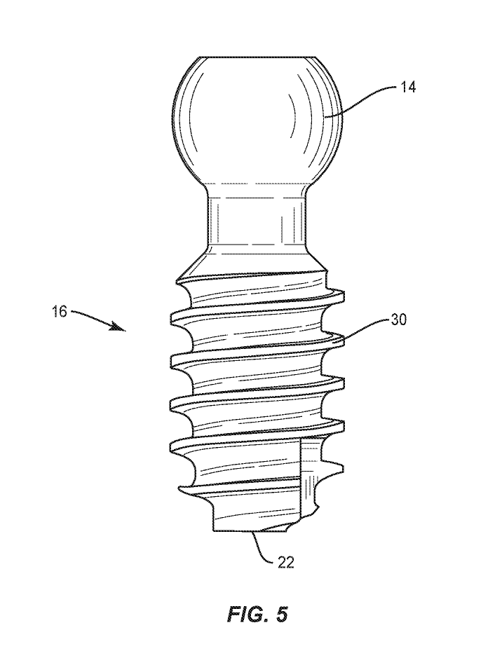

Shaft 16 includes an outer surface 26. Surface 26 includes a thread 30. Thread 30 extends along a length L of shaft 16 between end 20 and end 22, as shown in FIG. 3. In one embodiment, thread 30 is continuous along surface 26. In one embodiment, thread 30 may include a single thread turn or a plurality of discrete threads. In some embodiments, other penetrating elements may be located on shaft 16, such as, for example, a nail configuration, barbs, expanding elements, raised elements, ribs, and/or spikes to facilitate engagement of shaft 16 with tissue. In some embodiments, thread 30 may be self-tapping or intermittent.

Thread 30 includes a diameter D, as shown in FIG. 3. In some embodiments, shaft 16 includes a tapered thread portion 32. In some embodiments, shaft 16 includes a minor diameter D2. In some embodiments, bone screw 12 has a selected length L and a selected diameter D. In some embodiments, bone screw 12 has a selected length L and a selected diameter D employed with a minimally invasive lateral mass screw placement to reduce a risk of vertebral artery or nerve root injury.

In some embodiments, surgical system 10 can include one or a plurality of spinal implants, such as, for example, bone screws 12 provided in a kit or as a set having various configurations and dimensions. In some embodiments, the kit or set of bone screws 12 can be provided in various sizes from which a desired bone screw size and/or shape can be selected by a surgeon and/or selected for implant based on pre-operative planning or conditions encountered during surgery, as described herein.

In some embodiments, bone screw 12 is employed with a surgical method that aligns bone screw 12 with a direction of a force application to maximize a pullout force from along a trajectory of surgical pathway P to resist and/or prevent vertebral artery or nerve root injury. In some embodiments, bone screw 12 is aligned along a straight-in screw trajectory of surgical pathway P. In some embodiments, axis X1 of bone screw 12 is aligned with a direction of pullout force to provide an increased resistance to pullout. In some embodiments, bone screw 12 is employed with a cervical surgical procedure, such as, for example, a Roy-Camille technique. In some embodiments, bone screw 12 reduces a risk of vertebral artery and/or nerve root injury due to length L being configured to avoid breaching an anterior cortex of lateral mass tissue LM.

In some embodiments, bone screw 12 is configured with a selected length L based on a lateral mass length LML of a vertebral level of a cervical spine. In some embodiments, bone screw 12 includes selected length L in a range of 5.0 mm to 10.0 mm. In some embodiments, selected length L is in a range of 5.5 mm to 7.0 mm.

In some embodiments, diameter D is selected based on a lateral mass width LMW of a vertebral level of a cervical spine to maximize diameter D while maintaining the structural integrity of bone screw 12 when engaged with lateral mass tissue LM. A maximum diameter D and length L are selected to provide an increased pullout force. In some embodiments, selected diameter D is in a range of 4.5 mm to 8.0 mm. In some embodiments, selected diameter D is in a range of 6.0 mm to 8.0 mm.

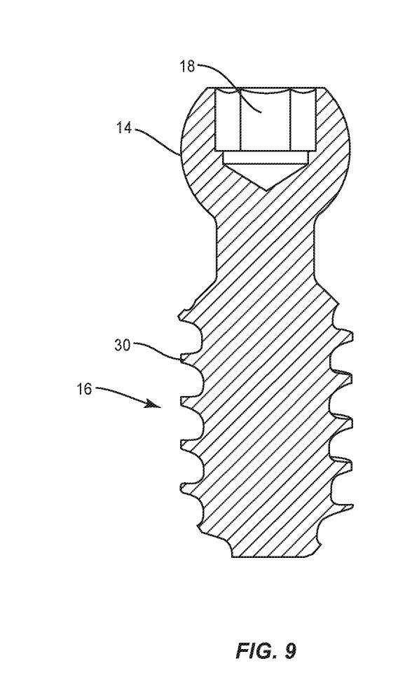

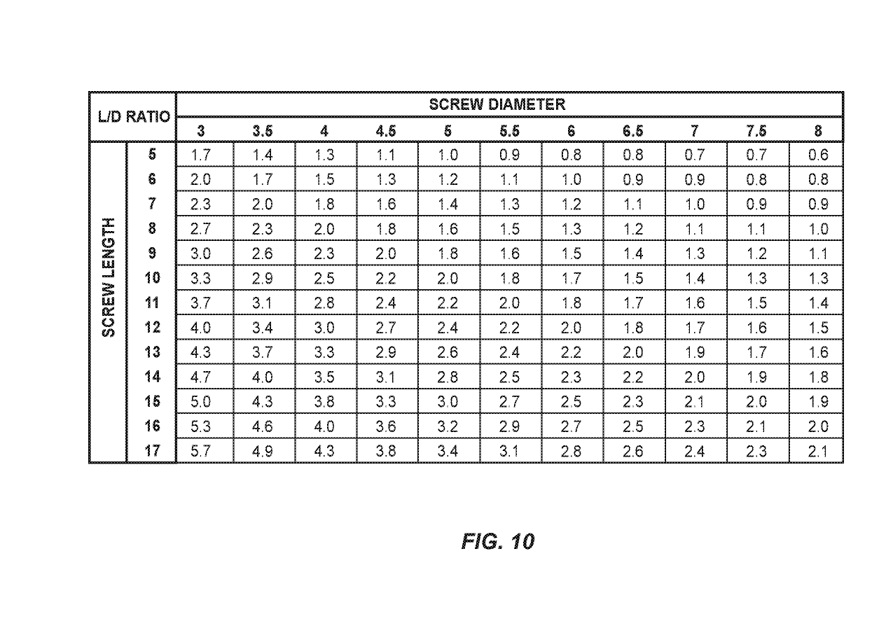

In some embodiments, as shown by the table in FIG. 10, selected length L and selected diameter D have a ratio L/D of bone screw 12 that is 2.0 or less. For example, bone screw 12 includes length L in a range of 5.0 mm to 10.0 mm and diameter D in a range of 4.5 mm to 8.0 mm such that ratio L/D of bone screw 12 is 2.0 or less. In some embodiments, bone screw 12 includes length L in a range of 6.0 mm to 8.0 mm and diameter D in a range of 5.5 mm to 7.0 mm such that ratio L/D of bone screw 12 is in a range of 0.9 to 1.5. In some embodiments, diameter D and/or length L are selected to provide a ratio L/D such that bone screw 12 resists and/or prevents damage to a vertebral artery and nerve roots while maintaining the structural integrity of bone screw 12. In some embodiments, ratio L/D is less than 2.0.

In some embodiments, length L of bone screw 12 is determined based on a patient anatomy, such as, for example, cervical lateral mass length LML and cervical lateral mass width LMW at a cervical level, such as, for example, between cervical levels C3-C7. In some cases, the lateral mass length LML and the lateral mass width LMW vary between male and female patients.

For example, a male patient anatomy can have a cervical lateral mass length of 11.7 mm and a cervical lateral mass width of 11.1 mm for a subaxial cervical spine at cervical level C3. In some examples, a male patient anatomy can have a cervical lateral mass length of 12.6 mm and a cervical lateral mass width of 11.4 mm for a subaxial cervical spine at cervical level C4. In some examples, a male patient anatomy can have a cervical lateral mass length of 12.9 mm and a cervical lateral mass width of 12.4 mm for a subaxial cervical spine at cervical level C5. In some examples, a male patient anatomy can have a cervical lateral mass length of 12.4 mm and a cervical lateral mass width of 12.8 mm for a subaxial cervical spine at cervical level C6. In some examples, a male patient anatomy can have a cervical lateral mass length of 9.8 mm and a cervical lateral mass width of 11.8 mm for a subaxial cervical spine at cervical level C7. In some examples, a female patient anatomy can have a cervical lateral mass length of 11.0 mm and a cervical lateral mass width of 10.0 mm for a subaxial cervical spine at cervical level C3. In some examples, a female can have a cervical lateral mass length of 11.5 mm and a cervical lateral mass width of 10.3 mm for a subaxial cervical spine at cervical level C4. In some examples, a female patient anatomy can have a cervical lateral mass length of 11.4 mm and a cervical lateral mass width of 11.0 mm for a subaxial cervical spine at cervical level C5. In some examples, a female patient anatomy can have a cervical lateral mass length of 11.1 mm and a cervical lateral mass width of 11.1 mm for a subaxial cervical spine at cervical level C6. In some examples, a female patient anatomy can have a cervical lateral mass length of 8.5 mm and a cervical lateral mass width of 10.3 mm for a subaxial cervical spine at cervical level C7.

In some embodiments, one or more bone screws 12 of the kit or set of surgical system 10 are selected based on the examples of male and female cervical measurements described herein, and dimensions of length L and/or diameter D are selected having length L in a range of 6.0 mm to 8.0 mm and diameter D in a range of 4.5 mm to 6.0 mm. In some embodiments, one or more bone screws 12 of the kit or set of surgical system 10 are selected based on the examples of male and female cervical measurements described herein, and length L and/or diameter D are selected such that bone screw 12 includes a ratio L/D of 2.0 or less, as shown by the table in FIG. 10. In some embodiments, length L and/or diameter D are selected such that bone screw 12 includes a ratio L/D of less than 2.0.

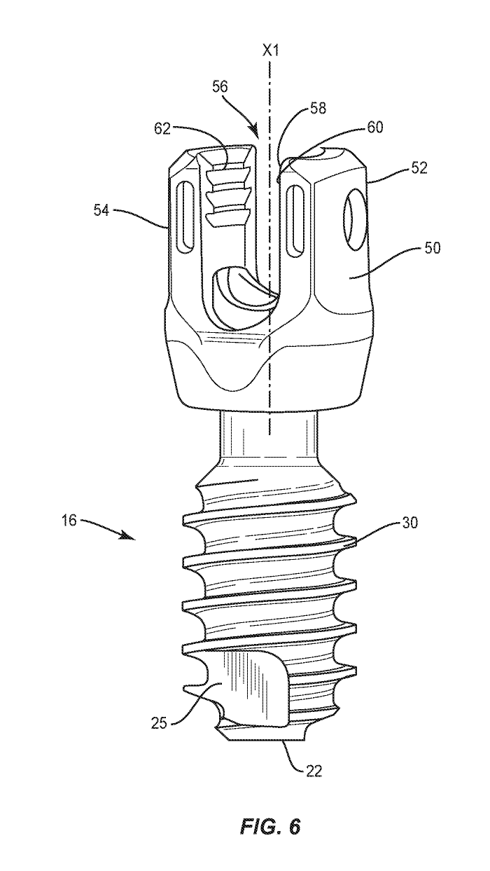

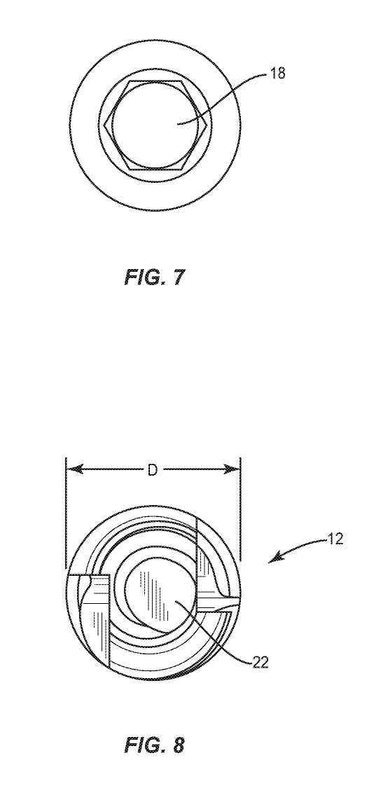

In some embodiments, head 14 is configured for connection with a receiver 50, as shown in FIG. 6. Receiver 50 extends along axis X1. Receiver 50 includes a pair of spaced apart arms 52, 54 that define an implant cavity 56 therebetween configured for disposal of a component of a spinal construct, such as, for example, a spinal rod (not shown). Arms 52, 54 each extend parallel to axis X1. In some embodiments, arms 52, 54 may be disposed at alternate orientations, relative to axis X1, such as, for example, transverse, perpendicular and/or other angular orientations such as acute or obtuse, coaxial and/or may be offset or staggered.

Cavity 56 is substantially U-shaped. In some embodiments, all or only a portion of cavity 56 may have alternate cross section configurations, such as, for example, closed, V-shaped, W-shaped, oval, oblong triangular, square, polygonal, irregular, uniform, non-uniform, offset, staggered, and/or tapered. Receiver 50 includes an inner surface 58 that includes a thread form 60 located adjacent arm 52 and a thread form 62 located adjacent arm 54. Thread forms 60, 62 are each configured for engagement with a coupling member, such as, for example, a setscrew (not shown), to retain a spinal construct, such as, for example, a spinal rod (not shown) within cavity 56. In some embodiments, surface 58 may be disposed with the coupling member in alternate fixation configurations, such as, for example, friction fit, pressure fit, locking protrusion/recess, locking keyway and/or adhesive. In some embodiments, all or only a portion of surface 58 may have alternate surface configurations to enhance engagement with the spinal rod and/or the setscrew such as, for example, rough, arcuate, undulating, mesh, porous, semi-porous, dimpled and/or textured. In some embodiments, receiver 50 may include alternate configurations, such as, for example, closed, open and/or side access.

In some embodiments, connection of head 14 with a receiver 50 forms various bone screw configurations, such as, for example, multi-axial screws, sagittal angulation screws, pedicle screws, mono-axial screws, uni-planar screws, fixed screws, anchors, tissue penetrating screws, conventional screws, expanding screws. In some embodiments, head 14 is configured for connection with a spinal implant, such as, for example, a plate. In some embodiments, head 14 is configured for connection with a post and/or connector that comprise a spinal construct and/or may be connected with a spinal rod. In some embodiments, head 14 may include and/or be integrally connected or monolithically formed with a post, and/or the post may be rotatable relative to axis X1.

In assembly, operation and use, surgical system 10, similar o the systems and methods described herein, includes a selected bone screw 12 and is employed with a surgical procedure for treatment of a condition or injury of an affected section of the spine. Surgical system 10 is utilized for treatment of a condition or injury of an affected section of the spine including cervical vertebrae V, such as, for example, lateral mass tissue LM of a cervical spine. In one embodiment, as shown in FIGS. 11-14, the components of surgical system 10 are employed with a method to treat vertebrae V.

In use, to treat a selected section of vertebrae V, a medical practitioner obtains access to a surgical site including vertebrae V, such as, for example, along a surgical pathway P. In some embodiments, surgical pathway P is oriented at angle α, as described herein, relative to a direct posterior surgical approach. In some embodiments, surgical system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway, which includes surgical pathway P, in alignment with a surgical approach; as described herein, for implantation of components of surgical system 10. A preparation instrument (not shown) can be employed to prepare tissue surfaces of vertebrae V, as well as for aspiration and irrigation of a surgical region.

Bone screw 12 is selected having a length L and a diameter D based on a cervical level of vertebrae V and patient criteria, such as, for example, male, female, lateral mass length LML and/or lateral width LMW. For example, lateral mass length LML and lateral width LMW of the patient is determined and diameter D and length L of bone screw 12 is selected from one or more bone screws 12 of the kit or set of surgical system 10. In some embodiments, diameter D and length L are selected based on the patient criteria and bone screw 12 is selected having a ratio L/D 2.0 or less to resist and/or prevent damage to vertebral arteries and/or nerves. In some embodiments, diameter D and length L are selected based on the patient criteria and bone screw 12 is selected having a ratio L/D less than 2.0 to resist and/or prevent damage to vertebral arteries and/or nerves.

Head 14 is engaged with a surgical instrument, such as, for example, a driver (not shown). In some embodiments, bone screw 12 is connected with a receiver 50 (FIG. 6). Bone screw 12 is translated such that axis X1 of shaft 16 is disposed along surgical pathway P. Surgical pathway P includes a trajectory disposed at angle α to penetrate lateral mass tissue LM. In some embodiments, angle α is in a range of 0 through 10 degrees relative to a direct posterior surgical approach. The driver is rotated causing bone screw 12 to translate axially within a pilot hole. Shaft 16 translates such that thread 30 engages lateral mass tissue LM and avoids penetrating the anterior cortex of lateral mass LM. As bone screw 12 is translated into lateral mass tissue LM, thread 30 engages lateral mass tissue LM. In some embodiments, length L of bone screw 12 resists and/or prevents damage to vertebral arteries and/or nerves and diameter D resists and/or prevents pullout while maintaining structural integrity.

Upon completion of a procedure, as described herein, the surgical instruments, assemblies and non-implanted components of surgical system 10 are removed and the incision(s) are closed. One or more of the components of surgical system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of surgical system 10. In some embodiments, surgical system 10 may include one or a plurality of rods, plates, connectors and/or bone fasteners for use with a single vertebral level or a plurality of vertebral levels.

In some embodiments, one or more bone fasteners, such as, for example, bone screw 12 may be engaged with tissue in various orientations, such as, for example, series, parallel, offset, staggered and/or alternate vertebral levels. In some embodiments, one or more bone spinal implants may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, clips, snaps, friction fittings, compressive fittings, expanding rivets, staples, nails, adhesives, posts, fixation plates and/or posts.

In one embodiment, surgical system 10 includes an agent, which may be disposed, packed, coated or layered within, on or about the components and/or surfaces of surgical system 10. In some embodiments, the agent may include bone growth promoting material, such as, for example, bone graft to enhance fixation of the components and/or surfaces of surgical system 10 with vertebrae. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
a first portion including a head;
a second portion including a threaded shaft having a length and a diameter, the threaded shaft including a length to diameter ratio of less than 2.0; and
a receiver configured to be coupled to the first portion, the receiver having spaced apart arms that define an implant cavity.

2. A spinal implant as recited in claim 1, wherein the ratio is in a range of less than 2.0 to 0.6.

1. l implant as recited in claim 1, wherein the ratio is in a range of 0.9 to 1.5.

4. A spinal implant as recited in claim 1, wherein the length is in a range of 5 mm to 10 mm.

5. A spinal implant as recited in claim 1, wherein the length is in a range of 6 mm to 8 mm.

6. A spinal implant as recited in claim 1, wherein the diameter is in a range of 4.5 mm to 8 mm.

7. A spinal implant as recited in claim 1, wherein the diameter is in a range of 4.5 mm to 6 mm.

8. A spinal implant as recited in claim 1, wherein the second portion includes a tapered tip.

9. A spinal implant as recited in claim 1, wherein the head comprises a post.

10. A spinal implant as recited in claim 1, wherein the receiver is rotatable relative to the head of the first portion.

11. A spinal implant as recited in claim 10, wherein the receiver includes a U-shaped channel configured to receive a spinal rod.

12. A spinal implant as recited in claim 1, wherein the second portion is configured to penetrate lateral mass tissue.

13. A spinal implant as recited in claim 1, wherein the second portion is configured to penetrate lateral mass tissue such that an axis of the second portion is disposed along a surgical pathway oriented at an angle in a range of 0 through 10 degrees relative to a direct posterior surgical approach.

14. A spinal implant comprising:
a first portion including a head;
a second portion including a threaded shaft having a length in a range of 5 mm to 10 mm and a diameter in a range of 4.5 mm to 8 mm; and
a receiver configured to connect to the first portion, the receiver having a U-shaped channel configured to receive a spinal rod.

15. A spinal implant as recited in claim 14, wherein the length is in a range of 5.5 mm to 7 mm.

16. A spinal implant as recited in claim 14, wherein the diameter is in a range of 6 mm to 8 mm.

17. A spinal implant as recited in claim 14, wherein the threaded shaft includes a length to diameter ratio of 2.0 or less.

18. A spinal implant as recited in claim 14, wherein the threaded shaft includes a length to diameter ratio in a range of 0.9 to 1.5.

19. A spinal implant as recited in claim 14, wherein the second portion is configured to penetrate lateral mass tissue such that an axis of the second portion is disposed along a surgical pathway oriented at an angle in a range of 0 through 10 degrees relative to a direct posterior surgical approach.

20. A spinal implant comprising:
a first portion including a head connected with an implant receiver; and
a second portion including a threaded shaft having a length in a range of 5 mm to 10 mm and a diameter in a range of 4.5 mm to 8 mm, the threaded shaft including a length to diameter ratio of 2.0 or less,
the second portion being configured to penetrate lateral mass tissue such that an axis of the second portion is disposed along a surgical pathway oriented at an angle in a range of 0 through 10 degrees relative to a direct posterior surgical approach.
